(19) United States
(12) Patent Application Publication
(10) Pub. No.: US 2024/0111719 A1
(43) Pub. Date: Apr. 4, 2024

(54) EXPOSING RISK TYPES OF BIOMEDICAL INFORMATION

(71) Applicant: Scinapsis Analytics Inc., dba BenchSci, Toronto (CA)

(72) Inventors: Luigi GENTILE, Mississauga (CA); Tom LEUNG, North York (CA); Casandra Savitri MANGROO, Pickering (CA)

(73) Assignee: Scinapsis Analytics Inc., dba BenchSci, Toronto (CA)

(21) Appl. No.: 17/958,196

(22) Filed: Sep. 30, 2022

(51) Int. Cl. G06F 16/14; G06F 3/0481

(52) U.S. Cl. CPC G06F 16/156; G06F 3/0481; G06F 3/04842

(57) ABSTRACT

A method exposes risk types of biomedical information. The method includes processing a plurality of result graphs to generate an evidence graph, in response to a query; presenting the evidence graph with a node corresponding to a result graph of the plurality of result graphs, wherein the result graph corresponds to a file; and presenting the node with a risk icon indicating a subset of files is identified with one or more risk types.

### BACKGROUND

Biomedical information includes literature and writings that describe evidence from experiments and research of biomedical science that provides the basis for modern medical treatments. Biomedical information is published in publications in physical or electronic form and may be distributed in electronic form using files. Databases of biomedical information provide access to the electronic forms of the publications. A challenge is for computing systems to automatically determine and display risks of adverse events that are identified in biomedical information.

### SUMMARY

In general, in one or more aspects, the disclosure relates to a method that exposes risk types of biomedical information. The method includes processing a plurality of result graphs to generate an evidence graph, in response to a query; presenting the evidence graph with a node corresponding to a result graph of the plurality of result graphs, wherein the result graph corresponds to a file; and presenting the node with a risk icon indicating a subset of files is identified with one or more risk types.

In general, in one or more aspects, the disclosure relates to a method that exposes risk types of biomedical information. The method includes receiving a selection of a node of an evidence graph generated from a plurality of result graphs; processing the selection of the node to present a popup window in response to the selection of the node; and presenting the popup window with a selection element indicating presence of one or more risk types in a subset of files corresponding to the node. The method further includes presenting the selection element with an enumeration of the subset of files.

In general, in one or more aspects, the disclosure relates to a system that exposes risk types of biomedical information. The system includes an evidence graph controller configured to generate an evidence graph; an interface controller configured to present a risk window; and an application executing on one or more servers. The application is configured for receiving a selection of a selection element; processing the selection of the selection element to present the risk window adjacent to the evidence graph using the interface controller; and presenting the risk window with a filter control, an export control, and a file list control. The application is further configured for presenting the file list control with one or more file summary tiles comprising a file summary tile; and presenting the file summary tile with an image from a figure from the file, a summary sentence generated from the file, and a summary risk label identifying a risk event of the file.

Other aspects of the invention will be apparent from the following description and the appended claims.

### BRIEF DESCRIPTION OF DRAWINGS

FIG. 1A, FIG. 1B, FIG. 1C, FIG. 1D, and FIG. 1E show diagrams of systems in accordance with disclosed embodiments.

FIG. 2A, FIG. 2B, FIG. 2C, FIG. 2D, and FIG. 2E show flowcharts in accordance with disclosed embodiments.

FIG. 3A, FIG. 3B, FIG. 4, FIG. 5, FIG. 6, FIG. 7A, FIG. 7B,

FIG. 7C, FIG. 7D, and FIG. 7E show examples in accordance with disclosed embodiments.

FIG. 8A and FIG. 8B show computing systems in accordance with disclosed embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure expose risk types identified in biomedical information (e.g., publications of biomedical literature). Different adverse events may present different types of risks including safety risks and efficacy risks. Embodiments of the disclosure analyze the biomedical information to generate result graphs and may then further analyze the biomedical information and the result graphs to identify risks from the biomedical information. The presence of a risk may be presented on the nodes of an evidence graph, in a summary of biomedical information, an in a view of the source of the biomedical information with the result graph for the source.

The figures show diagrams of embodiments that are in accordance with the disclosure. The embodiments of the figures may be combined and may include or be included within the features and embodiments described in the other figures of the application. The features and elements of the figures are, individually and as a combination, improvements to the technology of biomedical information processing and machine learning models. The various elements, systems, components, and steps shown in the figures may be omitted, repeated, combined, and/or altered as shown from the figures. Accordingly, the scope of the present disclosure should not be considered limited to the specific arrangements shown in the figures.

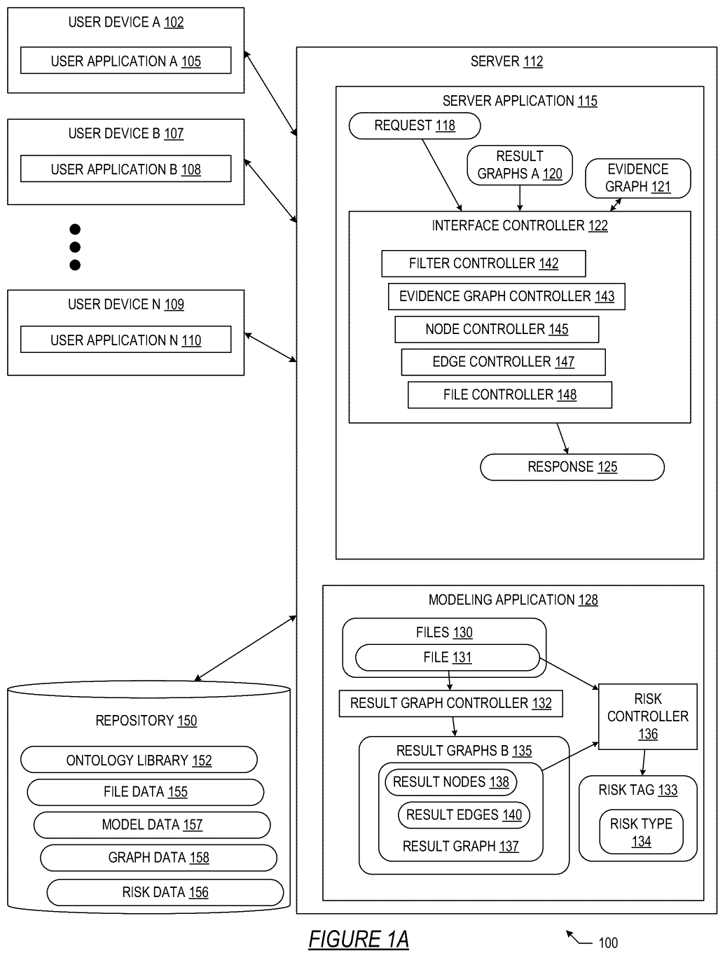

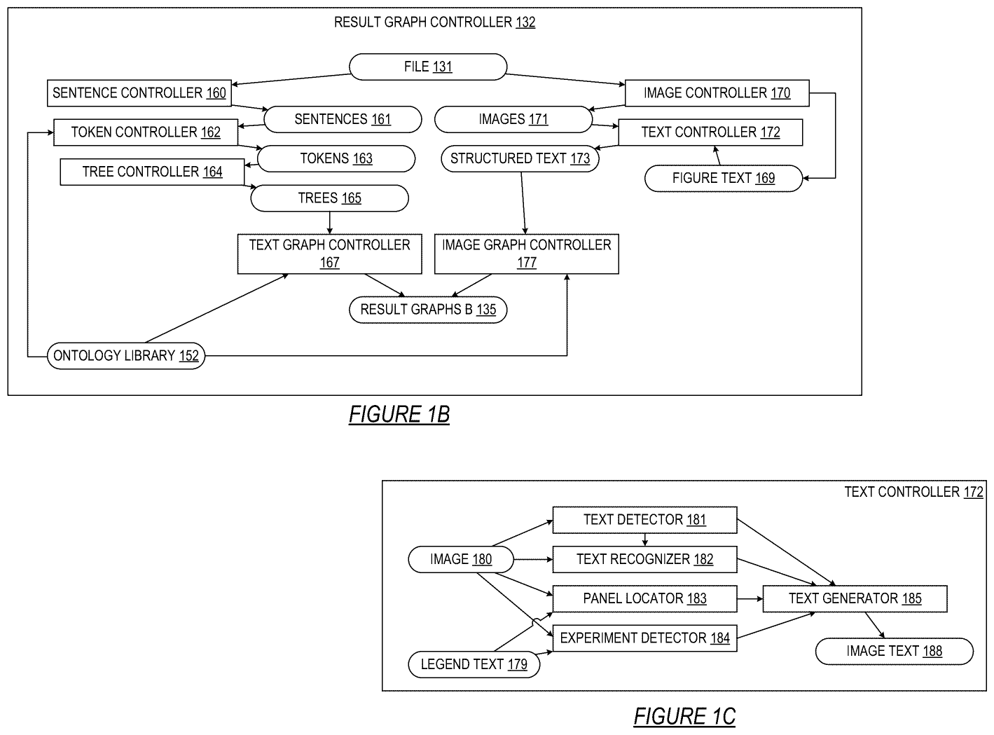

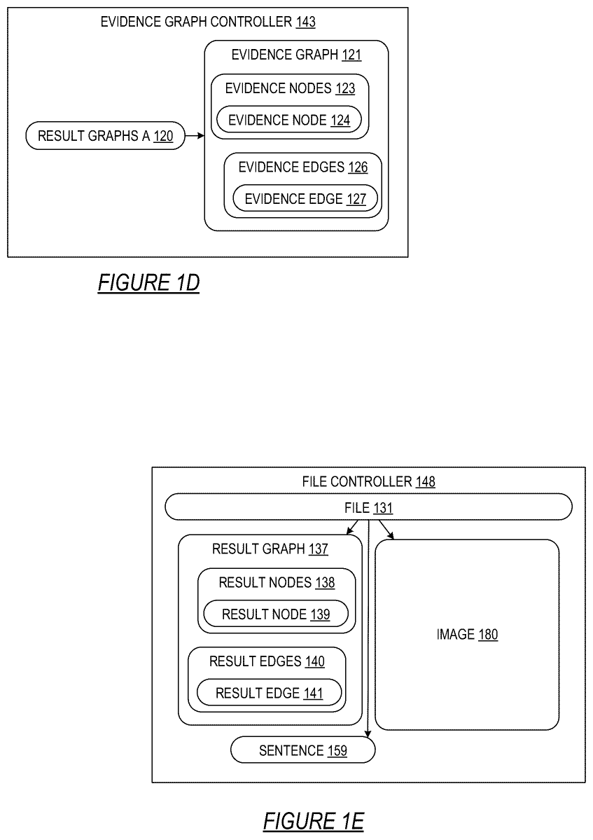

Turning to FIG. 1A, the system (100) implements evidence network navigation by converting biomedical information from files to result graphs and generating an evidence graph from the result graphs. The system (100) receives requests (e.g., the request (118)) and generates responses (e.g., the response (125)) using the result graphs A (120). The system (100) generates the result graphs A (120) from biomedical information (e.g., the files (131)) stored in the file data (155) using multiple machine learning and natural language processing models. The system (100) uses the result graphs A (120) to generate the evidence graph (121). The system (100) generates the response (125) using the evidence graph (121). The system (100) may display the evidence graph (121), the result graphs A (120), and the images from the files of the file data (155) to users operating the user devices A (102) and B (107) through N (109). The system (100) includes the user devices A (102) and B (107) through N (109), the server (112), and the repository (150).

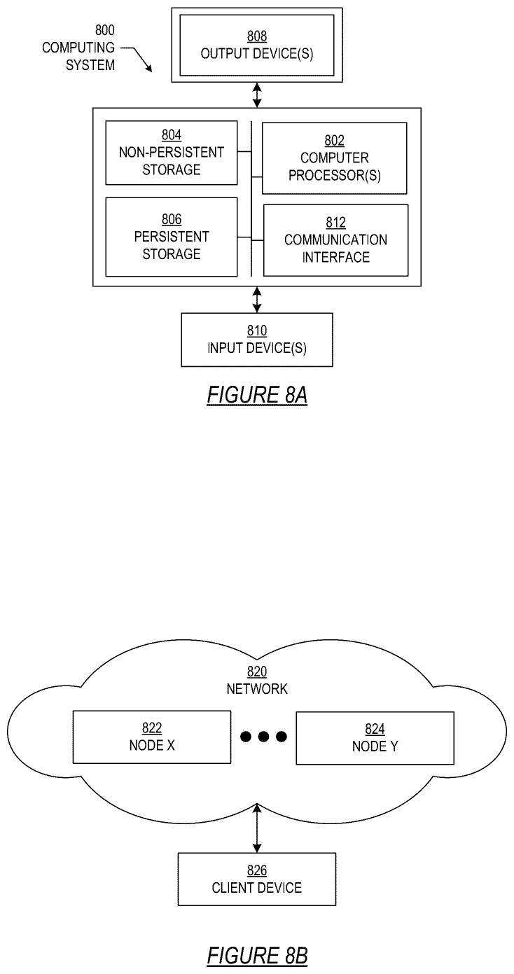

The server (112) is a computing system (further described in FIG. 8A). The server (112) may include multiple physical and virtual computing systems that form part of a cloud computing environment. In one embodiment, execution of the programs and applications of the server (112) is distributed to multiple physical and virtual computing systems in the cloud computing environment. The server (112) includes the server application (115) and the modeling application (128).

The server application (115) is a collection of programs that may execute on multiple servers of a cloud environment, including the server (112). The server application (115) receives the request (118) and generates the response (125) based on the result graphs A (120) using the evidence graph (121) and the interface controller (122). The server application (115) may host websites accessed by users of the user devices A (102) and B (107) through N (109) to view information from the evidence graph (121), the result graphs A (120), and the file data (155). The websites hosted by the server application (115) may serve structured documents (hypertext markup language (HTML) pages, extensible markup language (XML) pages, JavaScript object notation (JSON) files and messages, etc.). The server application (115) includes the interface controller (122), which processes the request (118) using the result graphs A (120) and the evidence graph (121).

The request (118) is a request from one of the user devices A (102) and B (107) through N (109). In one embodiment, the request (118) is a request for information about one or more entities defined in the ontology library (152), described in the file data (155), and graphed in the graph data (158). In one embodiment, the request (118) may specify additional filters for the list of entities. The structured text below (formatted in accordance with JSON) provides an example of entities that may be specified in the request (118) using key value pairs.

```
{
  “entity type”: “protein”,
  “entity”: “BRD9”,
}
{
  “entity type”: “disease”,
  “entity”: “breast cancer”,
}
```

The result graphs A (120) are generated with the modeling application (128), described further below. The result graphs A (120) includes nodes and edges in which the nodes correspond to text from the file data (155) and the edges correspond to semantic relationships between the nodes. The result graphs A (120) are directed graphs in which the edges identify a direction from one node to a subsequent node in the result graphs A (120). In one embodiment, the result graphs A (120) are acyclic graphs. The result graphs A (120) may be stored in the graph data (158) of the repository (150).

The evidence graph (121) is generated from the result graphs A (120) and may be stored in the graph data (158). Nodes of the evidence graph (121) represent entities from the ontology library (152). The edges of the evidence graph (121) identify files (e.g., publications) that record evidence of experiments related to the entities represented by the nodes of the evidence graph (121). For example, an edge may represent a file of a publication with a sentence (graphed as one of the result graphs A (120)) that includes the entities corresponding to the nodes of the edge. The edges of the evidence graph (121) may generate using the result graphs A (120). The evidence graph (121) may be stored in the graph data (158) of the repository (150).

The interface controller (122) is a collection of programs that may operate on the server (112). The interface controller (122) processes the request (118) using the result graphs A (120) and the evidence graph (121) to generate the response (125). In one embodiment, the interface controller (122) searches the graph data (158) to identify the result graphs A (120) (which may include some of the result graphs from the result graphs B (135)) that include information about the entities identified in the request (118). The interface controller (122) may update the evidence graph (121) using information from the request (118). For example, the request (118) may include filters to apply to the evidence graph (121), instructions to expand a node of the evidence graph (121), etc.

The filter controller (142) is a collection of programs that may operate on the server (112). The filter controller (142) processes the request (118) to apply filters to the evidence graph (121). The filters may filter the nodes and edges of the evidence graph (121). For example, the nodes may be filtered by entity names and entity types. The edges may be filtered by the number of experiments (e.g., the number of files with evidence including the entity of a node) sufficient to have an edge displayed between nodes of the evidence graph (121). A minimum number of experiments or files, a maximum number of experiments or files, etc., may be used as the filter. The edges may also be filtered by the type of publication (“published”, “preprint”, etc.) of the evidence in the file. In one embodiment, the filter controller (142) may process instructions from the request (118) that are generated responsive to a panel displayed on a left side of a user interface of one of the user application A (105) and user application B (108) through user application N (110).

The evidence graph controller (143) is a collection of programs that may operate on the server (112). The evidence graph controller (143) presents the evidence graph (121) in response to updates to the evidence graph (121). In one embodiment, the evidence graph controller (143) may generate HTML code that is transmitted to one of the user devices A (102) and B (107) through N (109), which display the evidence graph (121).

The node controller (145) is a collection of programs that may operate on the server (112). The node controller (145) updates the evidence graph (121) based on selections of a node from the request (118). In one embodiment, the node controller (145) may process instructions from the request (118) that are generated responsive to a popup menu displayed upon selection of a node from the evidence graph (121) using a user interface of one of the user applications A (105) and B (108) through N (110).

For example, the request (118) may include instructions to expand a node of the evidence graph (121) in response to a selection of a popup menu displayed on a node. The node controller (145) may add additional nodes to the evidence graph (121) to expand the selected node. The additional nodes may be located by searching the result graphs A (120) for graphs that include the entity represented by the selected node.

The edge controller (147) is a collection of programs that may operate on the server (112). The edge controller (147) may present summaries of information corresponding to one or more files based on a selection of an edge from the evidence graph (121). In one embodiment, the edge controller (147) may process instructions from the request (118) that are generated responsive to a panel displayed on a right side of a user interface of one of the user applications A (105) and B (108) through N (110).

The file controller (148) is a collection of programs that may operate on the server (112). In one embodiment, the file controller (148) may present information from a file (sentence, result graph, image, etc.).

The response (125) is generated by the interface controller (122) in response to the request (118) using the result graphs A (120). In one embodiment, the response (125) includes the evidence graph (121), which may be updated based on the request (118). The response (125) may further include one or more of the result graphs A (120) and information from the file data (155). Portions of the response (125) may be displayed by the user devices A (102) and B (107) through N (109) that receive the response (125).

The modeling application (128) is a collection of programs that may operate on the server (112). The modeling application (128) generates the result graphs B (135) from the files (130) using a result graph controller (132).

The files (130) include biomedical information and form the basis for the result graphs B (135). The files (130) include the file (131), which is the basis for the result graph (137). Each file includes multiple sentences and may include multiple images of evidence. The evidence may identify how different entities, defined in the ontology library (152), affect each other. For example, entities that are proteins may suppress or enhance the expression of other entities and affect the prevalence of certain diseases. Types of entities include proteins, genes, diseases, experiment techniques, chemicals, cell lines, pathways, tissues, cell types, organisms, etc. In one embodiment, nouns and verbs from the sentences of the file (131) are mapped to the result nodes (138) of the result graph (137). In one embodiment, the semantic relationships between the words in the sentences corresponding to the result nodes (138) are mapped to the result edges (140). In one embodiment, one file serves as the basis for multiple result graphs. In one embodiment, one sentence from a file may serve as the basis for one result graph.

The result graph controller (132) generates the result graphs B (135) from the files (130). The result graph controller (132) is a collection of programs that may operate on the server (112). For a sentence of the file (131), the result graph controller (132) identifies the result nodes (138) and the result edges (140) for the result graph (137).

The result graphs B (135) are generated from the files (130) and includes the result graph (137), which corresponds to the file (131). The result nodes (138) represent nouns and verbs from a sentence of the file (131). The result edges (140) identify semantic relationships between the words represented by the result nodes (138).

The risk controller (136) is a collection of programs that may operate on the server (112). The risk controller (136) generates the risk tag (133) from one or more of the file (131) and the result graph (137).

In one embodiment, the risk controller (136) uses risk signatures to identify risk events from one or more of the file (131) and the result graph (137). For example, a risk signature may identify a compound (a chemical, a protein, etc.) and a usage of the compound in a target (cell, tissue, organism, etc.). The compound and its usage may correspond to nodes of the result nodes (138). In one embodiment, the nodes for the compound and its usage are adjacent nodes.

In one embodiment, the risk controller (136) uses a machine learning model to identify risk events from the file (131). The machine learning models may take text from the file (131) (e.g., a sentence of a publication) as input and output a classification of a risk type. The modeling application (128) may process the output to generate the risk tag (133).

The risk tag (133) is a tag that identifies one or more of the file (131) and the result graph (137) as including a risk event. The risk tag may identify a term, phrase, or sentence from the file (131) that correspond to and describe the risk event. The risk tag may identify one or more of the result nodes (138) and result edges (140) that correspond to the risk event. The risk tag (133) includes the risk type (134).

The risk type (134) identifies the type of the risk of the risk tag (133). Risk types include safety risks and efficacy risks. A safety risk is a risk in which an adverse event was observed that affected the safety the cell, tissue, organ, organism, etc. For example, a safety risk may be identified in biomedical information describing an experiment in which a chemical introduced into a cell killed the cell. An efficacy risk is a risk in which an adverse event was observed that reduced the efficacy of a biomedical agent. For example, a chemical may be introduced that reduces the expression of a protein and reduces the efficacy of treatments with the protein.

The user devices A (102) and B (107) through N (109) are computing systems (further described in FIG. 8A). For example, the user devices A (102) and B (107) through N (109) may be desktop computers, mobile devices, laptop computers, tablet computers, server computers, etc. The user devices A (102) and B (107) through N (109) include hardware components and software components that operate as part of the system (100). The user devices A (102) and B (107) through N (109) communicate with the server (112) to access, manipulate, and view information including information from the graph data (158) and the file data (155). The user devices A (102) and B (107) through N (109) may communicate with the server (112) using standard protocols and file types, which may include hypertext transfer protocol (HTTP), HTTP secure (HTTPS), transmission control protocol (TCP), internet protocol (IP), hypertext markup language (HTML), extensible markup language (XML), etc. The user devices A (102) and B (107) through N (109) respectively include the user applications A (105) and B (108) through N (110).

The user applications A (105) and B (108) through N (110) may each include multiple programs respectively running on the user devices A (102) and B (107) through N (109). The user applications A (105) and B (108) through N (110) may be native applications, web applications, embedded applications, etc. In one embodiment, the user applications A (105) and B (108) through N (110) include web browser programs that display web pages from the server (112). In one embodiment, the user applications A (105) and B (108) through N (110) provide graphical user interfaces that display information stored in the repository (150).

As an example, the user application A (105) may be operated by a user and generate the request (118) to view information related to entities defined in the ontology library (152), described in the file data (155), and graphed in the graph data (158). Corresponding sentences and images from the file data (155) and graphs from the graph data (158) may be received in the response (125) and displayed in a user interface of the user application A (105).

As another example, the user device N (109) may be used by a developer to maintain the software applications hosted by the server (112) and train the machine learning models used by the system (100). Developers may view the data in the repository (150) to correct errors or modify the application served to the users of the system (100).

The repository (150) is a computing system that may include multiple computing devices in accordance with the computing system (800) and the nodes (822) and (824) described below in FIGS. 8A and 8B. The repository (150) may be hosted by a cloud services provider that also hosts the server (112). The cloud services provider may provide hosting, virtualization, and data storage services as well as other cloud services and to operate and control the data, programs, and applications that store and retrieve data from the repository (150). The data in the repository (150) includes the ontology library (152), the file data (155), the model data (157), and the graph data (158).

The ontology library (152) includes information that of the entities and biomedical terms and phrases used by the system (100). Multiple terms and phrases may be used for the same entity. The ontology library (152) defines types of entities. In one embodiment, the types include the types of protein/gene, chemical, cell line, pathway, tissue, cell type, disease, organism, etc. The ontology library (152) may store the information about the entities in a database, structured text files, combinations thereof, etc.

The file data (155) is biomedical information stored in electronic records. The biomedical information describes the entities and corresponding relationships that are defined and stored in the ontology library (152). The file data (155) includes the files (130). Each file in the file data (155) may include image data and text data. The image data includes images that represent the graphical figures from the files. The text data represents the writings in the file data (155). The text data for a file includes multiple sentences that each may include multiple words that each may include multiple characters stored as strings in the repository (150). In one embodiment, the file data (155) includes biomedical information stored as extensible markup language (XML) files, portable document files (PDFs). The file formats define containers for the text and images of the biomedical information describing evidence of biomedical experiments.

The model data (157) includes the data for the models used by the system (100). The models may include rules-based models and machine learning models. The machine learning models may be updated by training, which may be supervised training. The modeling application (128) may load the models from the model data (157) to generate the result graphs B (135) from the files (130).

The model data (157) may also include intermediate data. The intermediate data is data generated by the models during the process of generating the result graphs B (135) from the files (130).

The model data (157) may include the signatures, models, etc., used to identify the risk data (156). The signatures may define paths in result graphs of the graph data (158) that correspond to a risk event. The models may be machine learning models that identify risk events from biomedical information in the file data (155).

The graph data (158) is the data of the graphs (including the evidence graph (121) and the result graphs A (120) and B (135)) generated by the system. The graph data (158) includes the nodes and edges for the graphs. The graph data (158) may be stored in a database, structured text files, combinations thereof, etc.

The risk data (156) is the data that identifies risks of adverse events of the entities identified by the system. The risk data (156) includes risk tags (including the risk tag (133)).

Although shown using distributed computing architectures and systems, other architectures and systems may be used. In one embodiment, the server application (115) may be part of a monolithic application that implements evidence networks. In one embodiment, the user applications A (105) and B (108) through N (110) may be part of monolithic applications that implement evidence networks without the server application (115).

Turning to FIG. 1B, the result graph controller (132) processes the file (131) to generate the result graphs B (135). In one embodiment, the result graph controller (132) includes the sentence controller (160), the token controller (162), the tree controller (164), and the text graph controller (167) to process the text from the file (131) describing biomedical experiments. In one embodiment, the result graph controller (132) includes the image controller (170), the text controller (172), and the image graph controller (177) to process the figures from the file (131) that provide evidence for the conclusions of experiments.

The sentence controller (160) is a set of programs that operate to extract the sentences (161) from the file (131). In one embodiment, the sentence controller (160) cleans the text of the file (131) by removing markup language tags, adjusting capitalization, etc. The sentence controller (160) may split a string of text into substrings with each substring being a string that includes a sentence from the original text of the file (131). In one embodiment, the sentence controller (160) may filter the sentences and keep sentences with references to the figures of the file (131).

The sentences (161) are text strings extracted from the file (131). A sentence of the sentences (161) may be stored as a string of text characters. In one embodiment, the sentences (161) are stored in a list that maintains the order of the sentences (161) from the file (131). In one embodiment, the list may be filtered to remove sentences that do not contain a reference to a figure.

The token controller (162) is a set of programs that operate to locate the tokens (163) in the sentences (161). The token controller (162) may identify the start and stop of each token in a sentence

The tokens (163) identify the boundaries of words in the sentences (161). In one embodiment, a token (of the tokens (163)) may be a substring of a sentence (of the sentences (161)). In one embodiment, a token (of the tokens (163)) may be a set of identifiers that identify the locations of a start character and a stop character in a sentence. Each sentence may include multiple tokens.

The tree controller (164) is a set of programs that operate to generate the trees (165) from the tokens (163) of the sentences (161) of the file (131). In one embodiment, the tree controller (164) uses a neural network (e.g., the Berkeley Neural Parser)

The trees (165) are syntax trees of the sentences (161) to identify the parts of speech of the tokens (163) within the sentences (161). In one embodiment, the trees (165) are graphs with edges identifying parent child relationships between the nodes of a graph. In one embodiment, the nodes of a graph of a tree include a root node, intermediate nodes, and leaf nodes. The leaf nodes correspond to tokens (words, terms, multiword terms, etc.) from a sentence and the intermediate nodes identify parts of speech of the leaf nodes.

The text graph controller (167) is a set of programs that operate to generate the result graphs B (135) from the trees (165). In one embodiment, the text graph controller (167) maps the tokens (163) from the sentences (161) that represent nouns and verbs to nodes of the result graphs B (135). In one embodiment, the text graph controller (167) maps parts of speech identified by the trees (165) to the edges of the result graphs B (135).

In one embodiment, after generating an initial graph (of the result graphs B (135)) for a sentence (of the sentences (161)), the text graph controller (167) processes the graph using the ontology library (152) to identify the entities and corresponding entity types represented by the nodes of the graph. For example, a node of the graph may correspond to the token “BRD9”. The text graph controller (167) identifies the token as an entity defined in the ontology library (152) and identifies the entity type as a protein.

The image controller (170) is a set of programs that operate to extract figures from the file (131) to generate the images (171). The image controller also extracts the figure text (169) that corresponds to the images (171). In one embodiment, the image controller (170) may use rules and logic to identify the images and corresponding image text from the file (131). In one embodiment, the image controller (170) may use machine learning models to identify the images (171) and the figure text (169). For example, the file (131) may be stored in a page friendly format (e.g., a portable document file (PDF)) in which each page of the publication is stored as an image in a file. A machine learning model may identify pages that include figures and the locations of the figures on those pages. The located figures may be extracted as the images (171). Another machine learning model may identify the legend text that corresponds to and describes the figures, which is extracted as the figure text (169).

The images (171) are image files extracted from the file (131). In one embodiment, the file (131) includes the figures as individual image files that the image controller (170) converts to the images (171). In one embodiment, the figures of the file (131) may be contained within larger images, e.g., the image of a page of the file (131). The image controller (170) processes the larger images to extract the figures as the images (171).

The figure text (169) is the text from the file (131) that describes the images (171). Each figure of the file (131) may include legend text that describes the figure. The legend text for one or more figures of the file (131) is extracted as the figure text (169), which corresponds to the images (171).

The text controller (172) is a set of programs that operate to process the images (171) and the figure text (169) to generate the structured text (173). The text controller (172) is further described with FIG. 1C below.

The structured text (173) is strings of nested text with information extracted from the images (171) using the figure text (169). In one embodiment, the structured text (173) includes a JSON formatted string for each image of the images (171). In one embodiment, the structured text (173) identifies the locations of text, panels, and experiment metadata within the images (171). In one embodiment, the structured text (173) includes text that is recognized from the images (171). The structured text (173) may include additional metadata about the images (171). For example, the structured text may identify the types of experiments and the types of techniques used in the experiments that are depicted in the images (171).

The image graph controller (177) is a set of programs that operate to process the structured text (173) to generate one or more of the result graphs B (135). In one embodiment, the image graph controller (177) identifies text that corresponds to entities defined in the ontology library (152) from the structured text (173) and maps the identified text to nodes of the result graphs B (135). In one embodiment, the image graph controller (177) uses the nested structure of the structure text (173) to identify the relationships between the nodes of one or more of the result graphs B (135) and maps the relationships to edges of one or more of the result graphs B (135).

The result graphs B (135) are the graphs generated from the file (131) by the result graph controller (132). The result graphs B (135) include nodes that represent entities defined in the ontology library (152) and include edges that represent relationships between the nodes.

The ontology library (152) defines the entities that may be recognized by the result graph controller (132) from the file (131). The entities defined by the ontology library (152) are input to the token controller (162), the text graph controller (167), and the image graph controller (177), which identify the entities within the text and image extracted from the file (131).

Turning to FIG. 1C, the text controller (172) processes the image (180) and the corresponding legend text (179) to generate the image text (188). The text controller (172) may operate as part of the result graph controller (132) of FIG. 1B.

The image (180) is one of the images (171) from FIG. 1B. The image (180) includes a figure from the file (131) of FIG. 1B.

The legend text (179) is a string from the figure text (169) of FIG. 1B. The legend text (179) is the text from the legend of the figure that corresponds to the image (180).

The text detector (181) is a set of programs that operate to process the image (180) to identify the presence and location of text within the image (180). In one embodiment, the text detector (181) uses machine learning models to identify the presence and location of text. The location may be identified with a bounding box that specifies four points of a rectangle that surrounds text that has been identified in the image (180). The location of the text from the text detector (181) may be input to the text recognizer (182).

The text recognizer (182) is a set of programs that operates to process the image (180) to recognize text within the image (180) and output the text as a string. The text recognizer (182) may process a sub image from the image (180) that corresponds to a bounding box identified by the text detector (181). A machine learning model may then be used to recognize the text from the sub image and output a string of characters that correspond to the text within the sub image.

The panel locator (183) is a set of programs that operates to process the image (180) to identify the location of panels and subpanels within the image (180) or a portion of the image (180). A panel of the image (180) is a portion of the image, which may depict evidence of an experiment. The panels of the image (180) may contain subpanels to further subdivide information contained within the image (180). The image (180) may include multiple panels and subpanels that may identified within the legend text (179). The panel locator (183) may be invoked to identify the location for each panel (or subpanel) identified in the legend text (179). In one embodiment, the panel locator (183) outputs a bit array with each bit corresponding to a pixel from the image (180) and identifying whether the pixel corresponds to a panel.

The experiment detector (184); is a set of programs that operates to process the image (180) to identify metadata about experiments depicted in the image (180). In one embodiment, the experiment detector (184) processes the image (180) with a machine learning model (e.g., a convolutional neural network) that outputs a bounding box and a classification. In one embodiment, the bounding box may be an array of coordinates (e.g., top, left, bottom, right) in the image that identify the location of evidence of an experiment within the image. In one embodiment, the classification may be a categorical value that identifies experiment metadata, which may include the type of evidence, the type of experiment, or technique used in the experiment (e.g., graph, western blot, etc.).

The text generator (185) is a set of programs that operate to process the outputs from the text detector (181), the text recognizer (182), the panel locator (183), and the experiment detector (184) to generate the image text (188). In one embodiment, the text generator (185) creates a nested structure for the image text (188) based on the outputs from the panel locator (183), the experiment detector (184), and the text detector (181). For example, the text generator (185) may include descriptions for the panels, experiment metadata, and text from the image (180) in which the text and description of the experiment metadata may be nested within the description of the panels. Elements for subpanels may be nested within the elements for the panels.

The image text (188) is a portion of the structured text (173) of FIG. 1B that corresponds to the image (180). In one embodiment, the image text (188) uses a nested structure to describe the panels, experiment metadata, and text that are identified and located within the image (180).

Turning to FIG. 1D, the evidence graph controller (143) processes the result graphs A (120) to generate the evidence graph (121). The evidence graph controller (143) may operate as part of the interface controller (122) of FIG. 1A.

The evidence graph (121) stores relationships between entities from the ontology library (152) (of FIG. 1A) and corresponding files publications from the file data (155) (of FIG. 1A). The evidence graph (121) includes the evidence nodes (123) and the evidence edges (126).

The evidence nodes (123) (including the evidence node (124)) are the nodes of the evidence graph (121). The evidence nodes (123) represent the entities from the ontology library (152) (of FIG. 1A). For example, the evidence node (124) may correspond to the protein EPAS1.

The evidence edges (126) (including the evidence edge (127)) are the edges of the evidence graph (121). The evidence edges (126) represent an aggregation of the files that link the entities of the nodes of the evidence graph (121). The evidence edges (126) may be identified from the result graphs generated from files of the file data (155). For example, the evidence edge (127) may connect the evidence node (124) with another node of the evidence graph (121). The connection by the evidence edge (127) indicates that at least one file includes at least one result graph in which a path exists between the evidence node (124) and the other node of the evidence edge (127). In one embodiment, the path exists when at least one sentence from the file includes the alias names of the entities represented by the evidence node (124) and the other node. In one embodiment, the evidence graph controller (143) generates the evidence graph (121) in response to instructions from the request (118) (of FIG. 1A).

Turning to FIG. 1E, the file controller (148) presents information from the file (131). In one embodiment, the file controller (148) updates the response (125) (of FIG. 1A) to include the result graph (137), the image (180), and the sentence (159) from the file (131).

The file (131) is a file of biomedical information. The file (131) may correspond to the evidence edge (127) of the evidence graph (121). The file (131) is stored in the file data (155) (of FIG. 1A), includes the sentence (159) (from which the result graph (137) is generated) and includes the image (180), which corresponds to the sentence (159).

The result graph (137) is a result graph generated from the sentence (159) from the file (131). The result graph includes the result nodes (138) (including the result node (139)) and includes the result edges (140) (including the result edge (141)). The result node (139) may correspond to the same entity to which the evidence node (124) corresponds. The result edge (141) may identify a semantic relationship between the result node (139) and another result node from the result graph (137).

The sentence (159) is a sentence from the file (131). The sentence (159) is parsed to generate the result graph (137).

The image (180) is an image from the file (131). The image (180) comprises a figure from the file (131) that is referred to in the sentence (159).

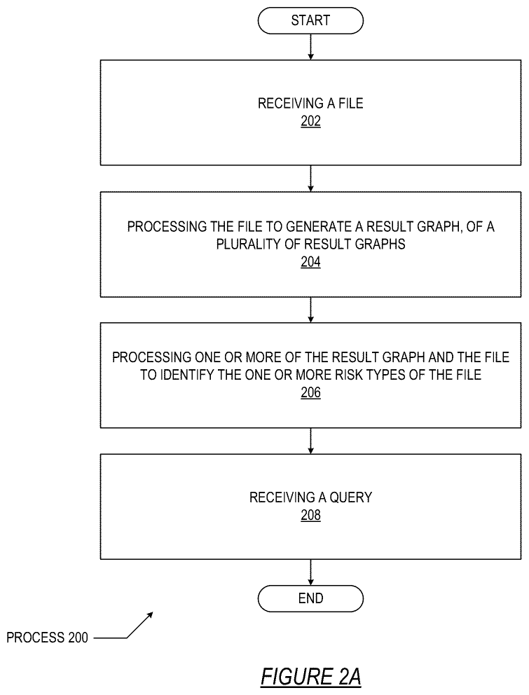

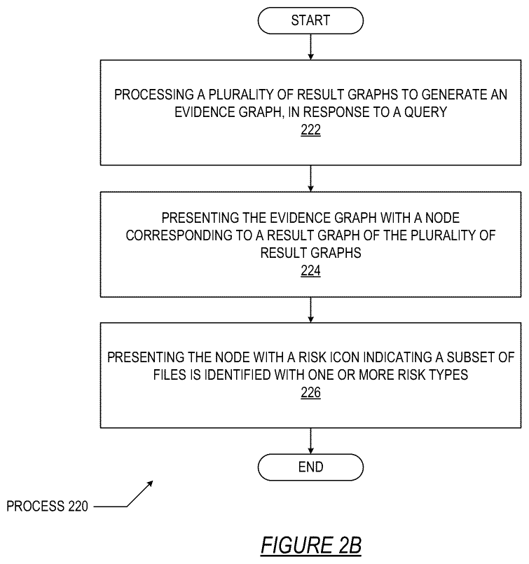

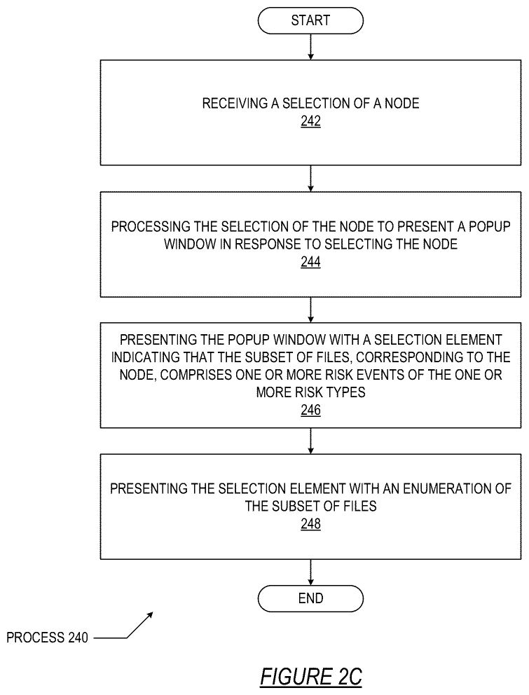

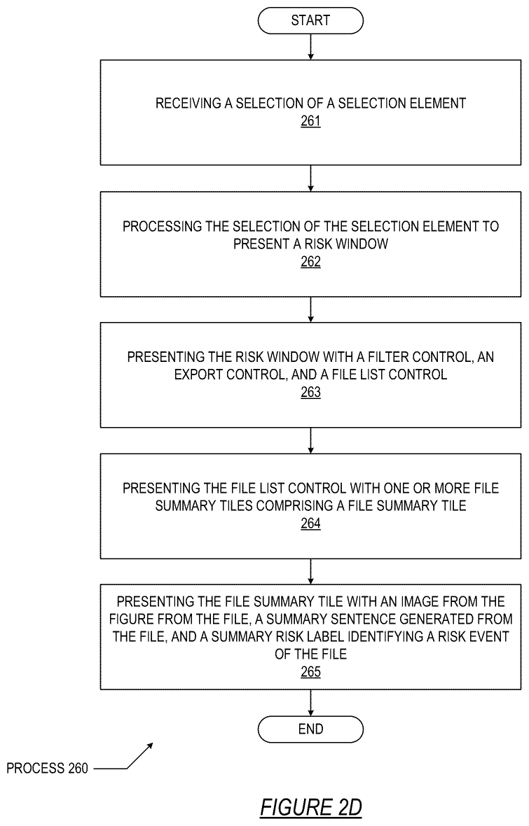

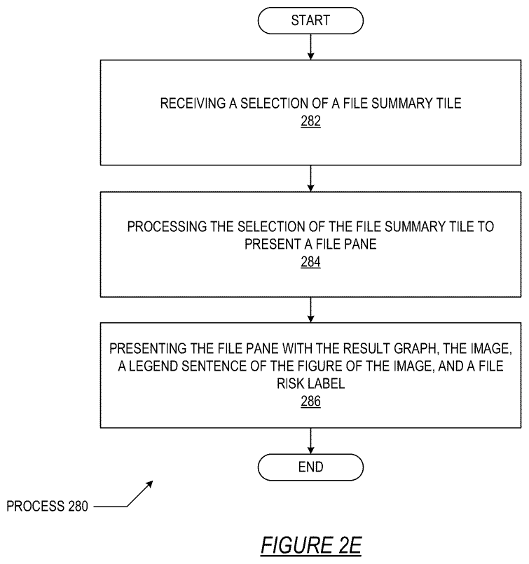

FIGS. 2A through 2E describe the processes (200), (220), (240), (260), and (280). The processes (200), (220), (240), (260), and (280) implement expose risk types of sources (e.g., files) of biomedical information.

Turning to FIG. 2A, the process (200) processes files. The files are sources of biomedical information. The process (200) may be performed by a computing system, such as the computing system (800) of FIG. 8A.

At Step 202, files are received. The files may be received from a database that stores the files. In one embodiment, the files may be received by scraping the files from a website. The files may be in the form of a structured document or set of documents. The structured document be a set of XML documents, a PDF document, a set of HTML documents, etc.

At Step 204, the file is processed to generate a result graph, of the plurality of result graphs. The file is processed using natural language processing and machine learning models. For example, the sentences of the file may be processed by a text graph controller to generate a result graph from one or more of the sentences that is matched to a figure of the file. A result graph may be generated for each file. Each result graph may correspond to a figure from the file corresponding to the result graph.

At Step 206, one or more of the result graph and the file are processed to identify the one or more risk types of the file. One or more rules or machine learning models may be used to identify the risk type.

In one embodiment, a rule may specify the nodes of a path that, if present in the result graph, identifies a type of risk. Different nodes and paths may identify different types of risk.

In one embodiment, a machine learning model may take the sentence as an input and output a classification that identifies a type of risk. The sentence may be tokenized using an algorithm (e.g., word2vec) and then input to the machine learning model. The machine learning model may output a classification vector with an element for each type of classification (e.g., a safety element and an efficacy element) and may include a null element for when the sentence does not include words, terms, phrases, that identify a risk event (i.e., an adverse event). The element of the classification vector with the largest value may be used as the output of the machine learning model to identify the type, if any, of a risk described by the file.

At Step 208, a query is received. The query may be received from a user device being operated by a user. The query may identify an entity (protein, chemical, disease, etc.) that the user is interested in researching.

Turning to FIG. 2B, the process (220) exposes risks using an evidence graph. The process (220) may be performed by a computing system, such as the computing system (800) of FIG. 8A.

At Step 222, result graphs are processed to generate an evidence graph. The evidence graph is generated in response to a query. The query identifies an entity that a user is researching. The evidence graph includes nodes that represent the entities for which a file (including a figure with a corresponding result graph) exists linking the entity of a node to the entity specified by the query.

At Step 224, the evidence graph is presented. The evidence graph is presented with a node corresponding to a result graph of the plurality of result graphs. The result graph corresponds to a file. In one embodiment, the evidence graph is presented by transmitting the evidence graph to a user device that displays the evidence graph in a user interface.

At Step 226, the node is presented with a risk icon indicating a subset of files is identified with one or more risk types. The subset of files is a subset of files that have been analyzed by the system. The subset of files includes the file, and the file includes a word corresponding to a node presented in the evidence graph.

In one embodiment, a central node represents the entity identified in the query and surrounding nodes represent entities identified from searching through the result graphs generated by the system for the entity of the central node. The central node and a surrounding node are connected by an edge to represent that at least one file has a corresponding result graph that includes nodes for the entities represented by the central node and the surrounding node. The surrounding node is presented with the risk icon to identify that at least one of the files identified by the edge between the central node and the surrounding node includes a risk event.

Turning to FIG. 2C, the process (240) exposes risks using a popup window. The process (240) may be performed by a computing system, such as the computing system (800) of FIG. 8A.

At Step 242, a selection of the node is received. The node may be selected in response to a user interacting with a user interface. For example, the user may right click on the node. The right click event may be transferred to the server, which then response to the event. In one embodiment, the event may be handled locally on the user device with information that is part of a monolithic application or with information that has already been transferred to the user device from the server.

At Step 244, the selection of the node is processed to present a popup window in response to selecting the node. The popup window may be displayed by the user device to the user. The location of the popup window may correspond to the location of the node. For example, the popup window may be displayed adjacent to the node, overlaid onto the node, etc.

At Step 246, the popup window is presented with a selection element indicating that the subset of files, corresponding to the node, includes one or more risk events of one or more risk types. The subset of files is a subset of the files analyzed by the system that correspond to the result graphs generated by the system.

At Step 248, the selection element is presented with an enumeration of the subset of files. The selection element may include text that identifies the cumulative number of files, the number of risk events, etc., of the subset of files. In one embodiment, the selection element may enumerate the number of risk events for each risk type and may use a different color for different types of risk.

Turning to FIG. 2D, the process (260) exposes risks using a risk window. The process (220) may be performed by a computing system, such as the computing system (800) of FIG. 8A.

At Step 261, a selection of the selection element is received. In one embodiment, the selection is received by a browser application that transmits the selection to a server.

At Step 262, the selection of the selection element is processed to present a risk window. The risk window may be displayed adjacent to the window displaying the node that was selected from the evidence graph.

At Step 263, the risk window is presented with a filter control, an export control, and a file list control. The filter control, the export control, and the file list control may be presented in a vertical alignment with the filter control above the export control and the file list control and the export control above the file list control.

The filter control includes user interface elements used to filter the files displayed in the file list control. For example, the files may be filtered by the type of risk (e.g., safety or efficacy), date of publication, type of publication (journal or pre-print), etc. In response to interaction with the filter control, the file list control may be updated using one or more selections of the one or more risk types.

Interaction with the export control exports the list of files with risk events that may be displayed with the file list control. In response to interaction with the export control, descriptions of files listed in the file list control are exported to a structured file. For example, the structured file may be a common separated value (CSV) file that includes a table with rows for each file and columns for data from the files. The columns may include the title of the publication corresponding to the file, the title of the article, the date of publication, etc.

At Step 264, the file list control is presented with one or more file summary tiles. A file summary tile presents information from a file related to the risk events detected by the system for the node selected by the user.

At Step 265, the file summary tile is presented with an image from the figure from the file, a summary sentence generated from the file, and a summary risk label identifying a risk event of the file. The image is from a figure that corresponds to evidence of the experiment that includes the risk event from the file. In one embodiment, the summary sentence is a portion of a sentence from the file that describes the image from the figure and may include a term or phrase that triggered the identification of the risk event.

Turning to FIG. 2E, the process (280) exposes risks using a result graph. The process (280) may be performed by a computing system, such as the computing system (800) of FIG. 8A.

At Step 282, a selection of the file summary tile is received. The selection may be from a user clicking or tapping on the file summary tile.

At Step 284, the selection of the file summary tile is processed to present a file pane. The file pane may be a new window that is displayed over the window with the evidence graph.

At Step 286, the file pane is presented with the result graph, the image, a legend sentence of the figure of the image, and a file risk label. The image corresponds to the result graph and shows the experimental evidence for the experiment corresponding to the result graph. The legend sentence may be text from the file provided for the figure from which the image is derived. In one embodiment, the file risk level may identify the type of risk that the system identified in the file.

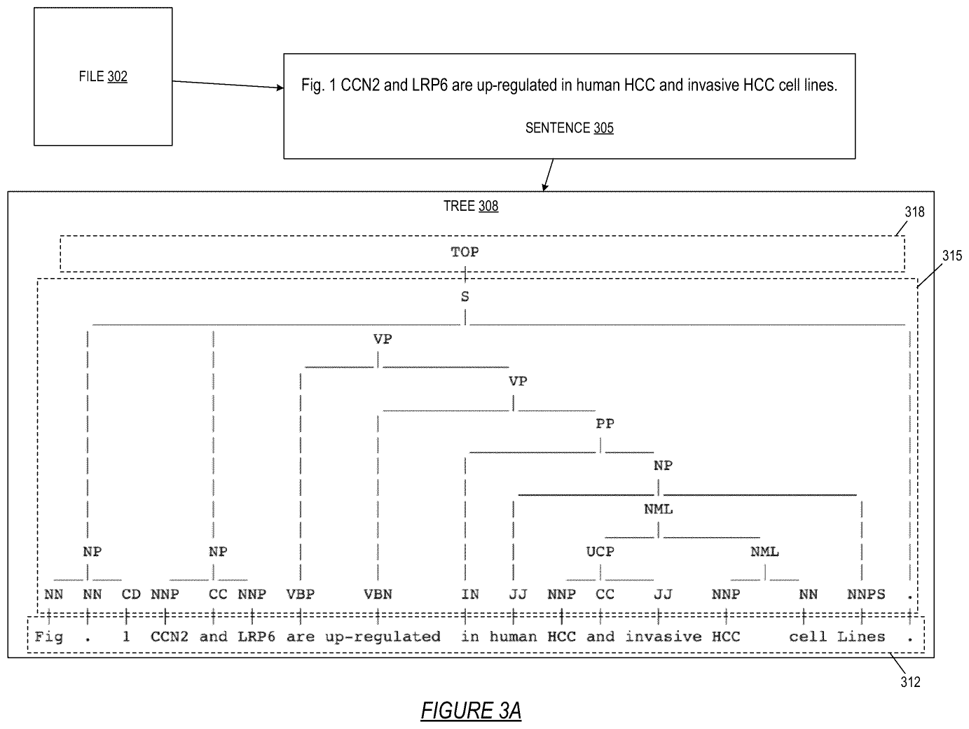

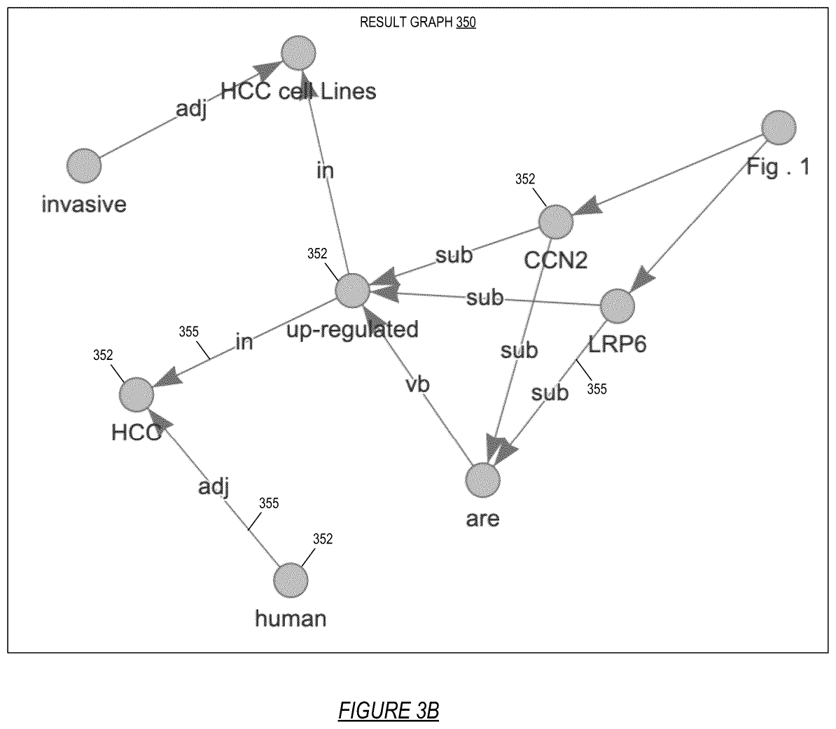

Turning to FIG. 3A, the file (302) is shown from which the sentence (305) is extracted, which is used to generate the tree (308), which is used to generate the graph (350) (of FIG. 3B). The file (302), the sentence (305), the tree (308), and the result graph (350) (of FIG. 3B) may be stored as electronic files, transmitted in messages, and displayed on a user interface.

The file (302) is a collection of biomedical information, which may include, but is not limited to, a writing of biomedical literature with sentences and figures stored as text and images. Different sources of biomedical information may be used. The file (302) is processed to extract the sentence (305).

The sentence (305) is a sentence from the file (302). The sentence (305) is stored as a string of characters. In one embodiment, the sentence (305) is tokenized to identify the locations of entities within the sentence (305). For example, the entities recognized from the sentence (305) may include “CCN2”, “LRP6”, “HCC”, and “HCC cell lines”. The sentence (305) is processed to generate the tree (308).

The tree (308) is a data structure that identifies semantic relationships of the words of the sentence (305). The tree (308) includes the leaf nodes (312), the intermediate nodes (315), and the root node (318).

The leaf nodes (312) correspond to the words from the sentence (305). The leaf nodes have no child nodes. The leaf nodes have parent nodes in the intermediate nodes (315).

The intermediate nodes (315) include values that identify the parts of speech of the leaf nodes (312). The intermediate nodes (315) having leaf nodes as direct children nodes identify the parts of speech of the words represented by the leaf nodes. The intermediate nodes (315) that do not have leaf nodes as direct children nodes identify the parts of speech of groups of one or more words, i.e., phrases, of the sentence (305).

The root node (318) is the top of the tree (308). The root node (318) has no parent node.

Turning to FIG. 3B, the result graph (350) is a data structure that represents the sentence (305) (of FIG. 3A). The result graph (350) may be generated from the sentence (305) and the tree (308). The nodes of the result graph (350) represent nouns (e.g., “CCN2”, “HCC”, etc.) and verbs (e.g., “up-regulated”, “are”, etc.) from the sentence (305). The edges (355) identify semantic relationships (e.g., subject “sub”, verb “vb”, adjective “adj”) between the words of the nodes (352) of the sentence (305) (of FIG. 3A). The result graph (350) is a directed acyclic graph.

Turning to FIG. 4, the image (402) is shown from which the structured text (405) is generated, which is used to generate the result graph (408). The image (402), the structured text (405), and the result graph (408) may be stored as electronic files, transmitted in messages, and displayed on a user interface.

The image (402) is a figure from a file (e.g., the file (302) of FIG. 3A, which may be from a biomedical publication). In one embodiment, the image (402) is an image file that is included with or as part of the file (302) of FIG. 3A. In one embodiment, the image (402) is extracted from an image of a page of a publication stored as the file (302) of FIG. 3A. The image (402) includes three panels labeled “A”, “B”, and “C”. The “B” panel includes three subpanels labeled “BAF complex”, “PBAF complex”, and “ncBAF complex”. The image (402) is processed to recognize the locations of the panels, subpanels, and text using machine learning models. After being located, the text from the image is recognized and stored as text (i.e., strings of characters). The panel, subpanel, and text locations along with the recognized text are processed to generate the structured text (405).

The structured text (405) is a string of text characters that represents the image (402). In one embodiment, the structured text (405) includes nested lists that form a hierarchical structure patterned after the hierarchical structure of the panels, subpanels, and text from the image (402). The structured text (405) is processed to generate the result graph (408).

The result graph (408) is a data structure that represents the figure, corresponding to the image (402), from a file (e.g., the file (302) of FIG. 3A). The result graph (408) includes nodes and edges. The nodes represent nouns and verbs identified in the structured text (405). The edges may represent the nested relationships between the panels, subpanels, and text of the image (402) described in the structured text (405).

Turning to FIG. 5, the tagged sentence (502) is generated from a sentence and used to generate the updated result graph (505). The tagged sentence (502) and the updated result graph (505) may be stored as electronic files, transmitted in messages, and displayed on a user interface.

The tagged sentence (502) is a sentence from a file that has been processed to generate the updated result graph (505). The sentence from which the tagged sentence is derived is input to a model to tag the entities in the sentence to generate the tagged sentence (502). The model may be a rules-based model, an artificial intelligence model, combinations thereof, etc.

As an example, the underlined portion (“INSR and PIK3R1 levels were not altered in TNF-alpha treated myotubes”) is tagged by the model. The terms “INSR”, “PIK3R1”, and “TNF-alpha” may be tagged as one type of entity that is presented as green when displayed on a user interface. The term “not” is tagged and may be displayed as orange. The terms “altered” and “treated” are tagged and may be displayed as pink. The term “myotubes” is tagged and may be displayed as red. After being identified in the sentence, the tags may be applied to the graph to generate the updated result graph (505).

The updated result graph (505) is an updated version of a graph of the sentence used to generate the tagged sentence (502). The graph is updated to label the nodes of the graph with the tags from the tagged sentence. For example, the nodes corresponding to “INSR” and “PIK3R1” are labeled with tags identified in the tagged sentence and may be displayed as green. The node corresponding to “altered” is tagged and displayed as pink. The node corresponding to “myotubes” is tagged and displayed as red.

Turning to FIG. 6, the user interface (600) displays information from a file, which may be a publication of biomedical literature. Different sources of files may be used. The user interface (600) may display the information on a user device after receiving a response to a request for the information transmitted to a server application. For example, the request may be for a publication that includes evidence linking the proteins “BRD9” and “A549”. The user interface displays the header section (602), the summary section (605), and the figure section (650).

The header section (602) includes text identifying the file being displayed. In one embodiment, the text in the header section (602) includes the name of the publication, the name of the author, the title of the publication, etc., which may be extracted from the file. Additional sources of information may be used, including patents, ELN data, summary documents, portfolio documents, scientific data in raw/table form, presentations, etc., and similar information may be extracted.

The summary section (605) displays information from the text of the file identified in the header section (602). The summary section (605) includes the graph section (608) and the excerpt section (615).

The graph section (608) includes the result graphs (610) and (612). the result graphs (610) and (612) were generated from the sentence displayed in the excerpt section (615). The result graph (612) shows the link between the proteins “BRD9” and “A549”, which conforms to the request that prompted the response with the information displayed in the user interface (600).

The excerpt section (615) displays a sentence from the file identified in the header section (602). The sentence in the excerpt section (615) is the basis from which the result graphs (610) and (612) were generated by tokenizing the sentence, generating a tree from the tokens, and generating the result graphs (610) and (612) from the tokens and tree.

The figure section (650) displays information from the figures of the file identified in the header section (602). The figure section (650) includes the image section (652) and the legend section (658).

The image section (652) displays the image (655). The image (655) was extracted from the file identified in the header section (602). The image (655) corresponds to the text from the legend section (658). The image (655) corresponds to the result graph (612) because the sentence shown in the excerpt section (615) identifies the figure (“Fig EV1A”) that corresponds to the image (655).

The legend section (658) displays the text of the legend that corresponds to the figure of the image (655). In one embodiment, the text of the legend section (655) may be processed to generate one or more graphs from the sentence in the legend section (658).

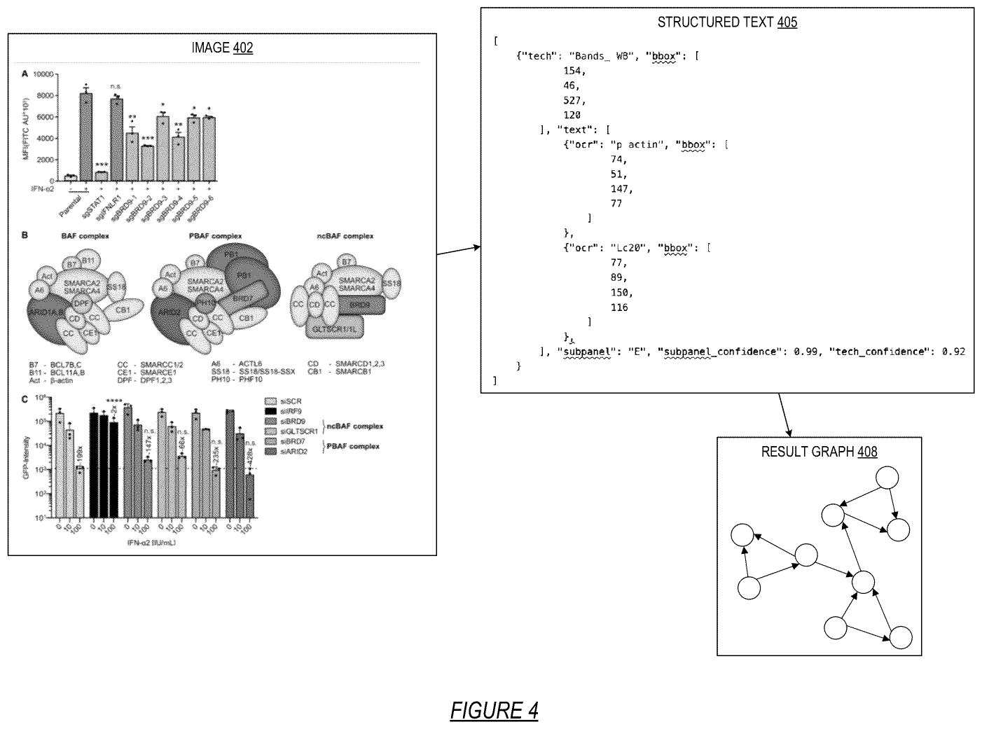

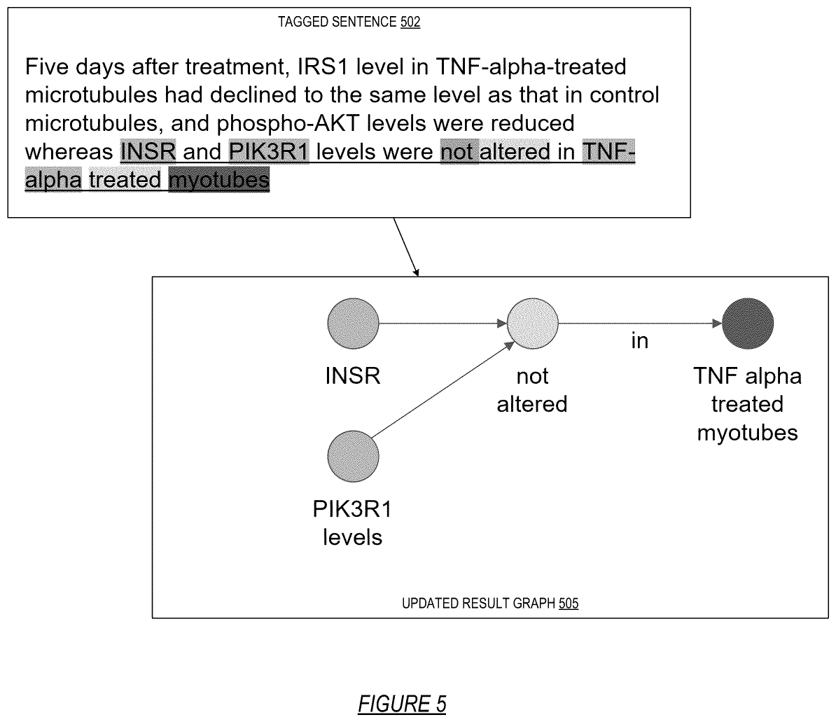

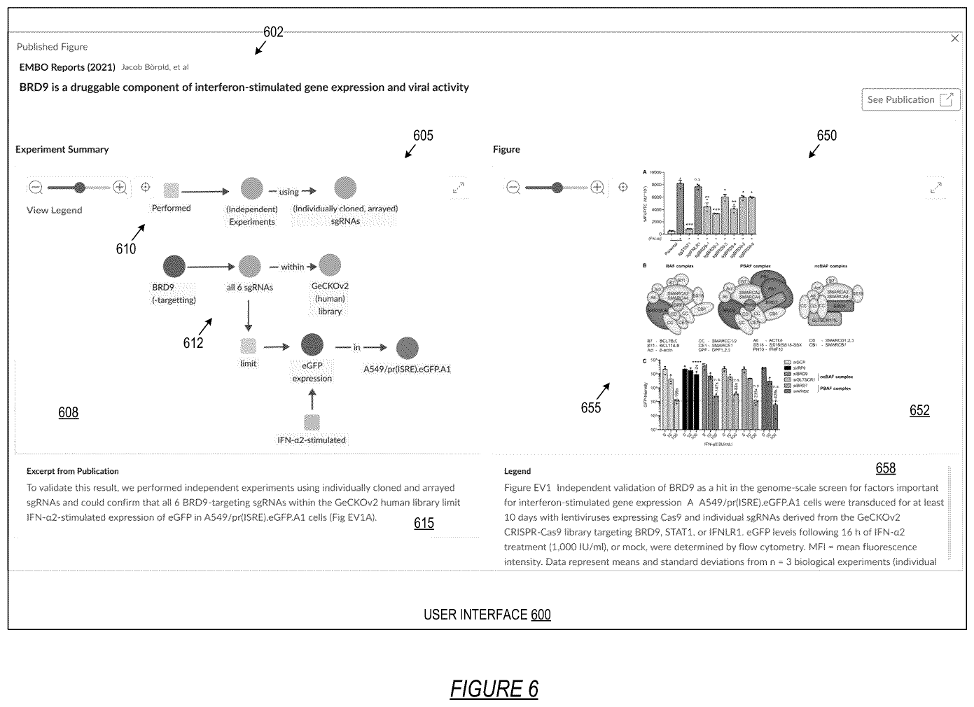

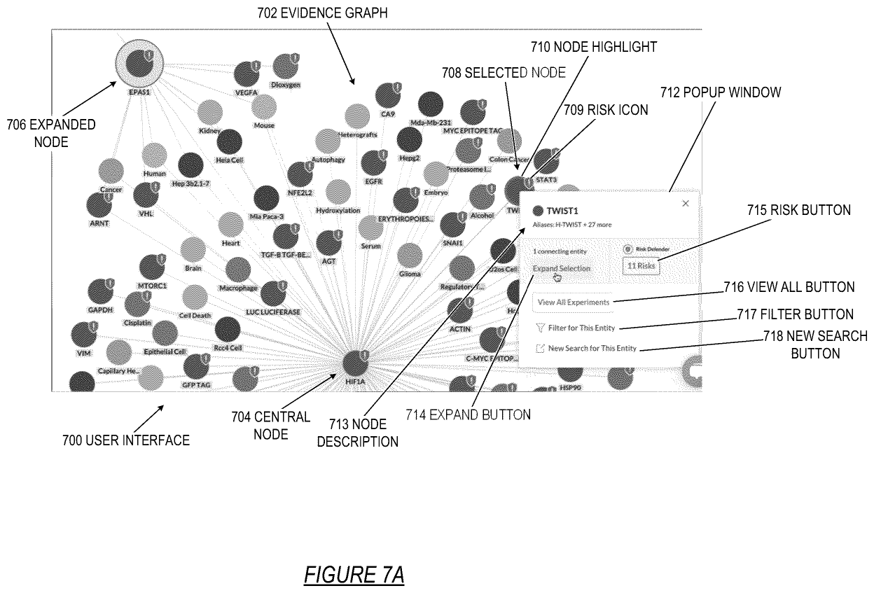

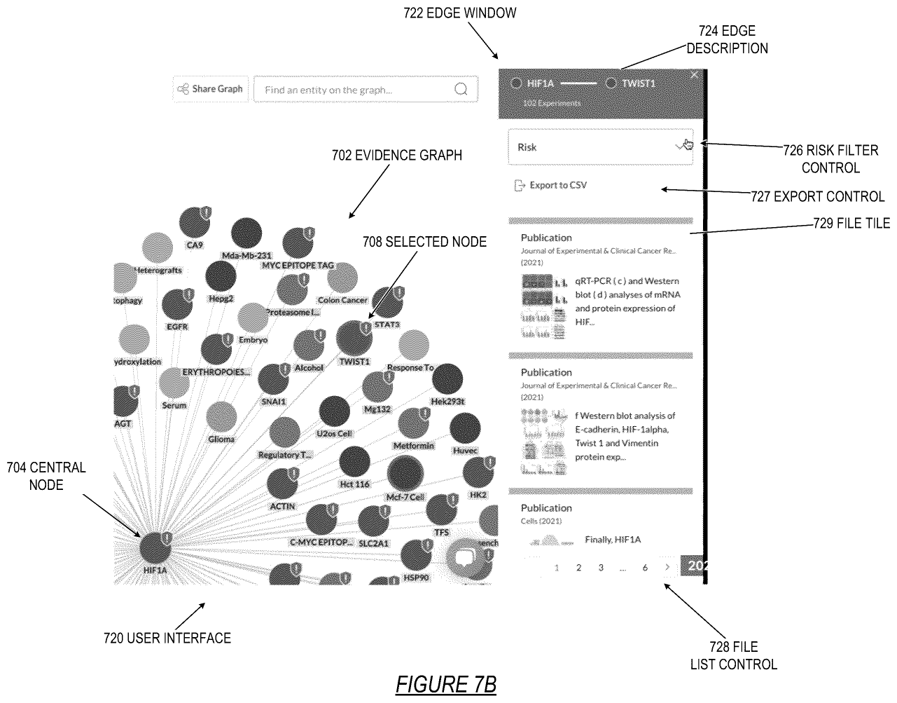

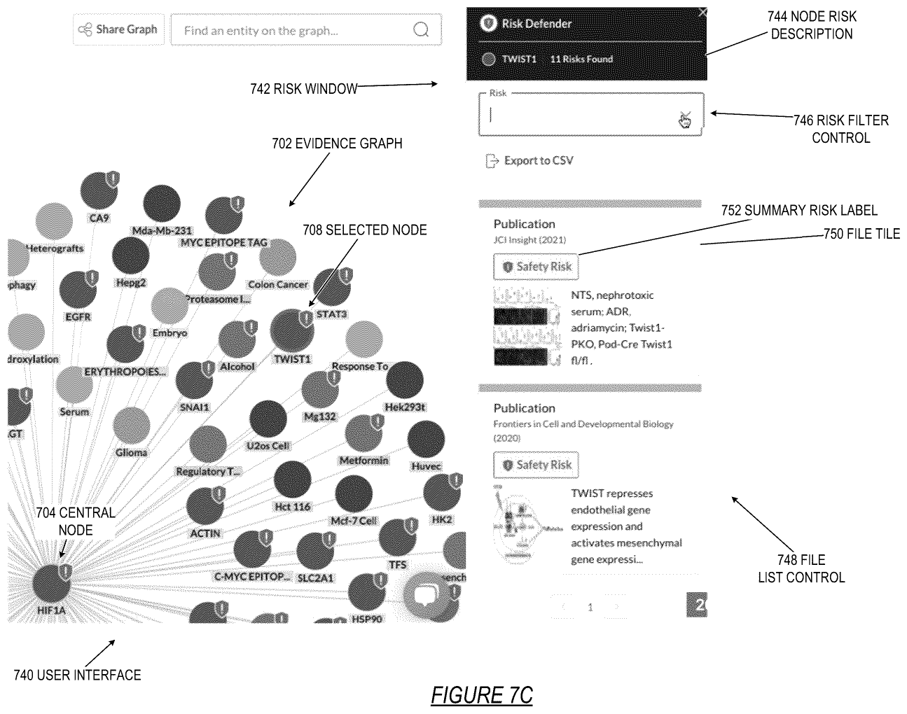

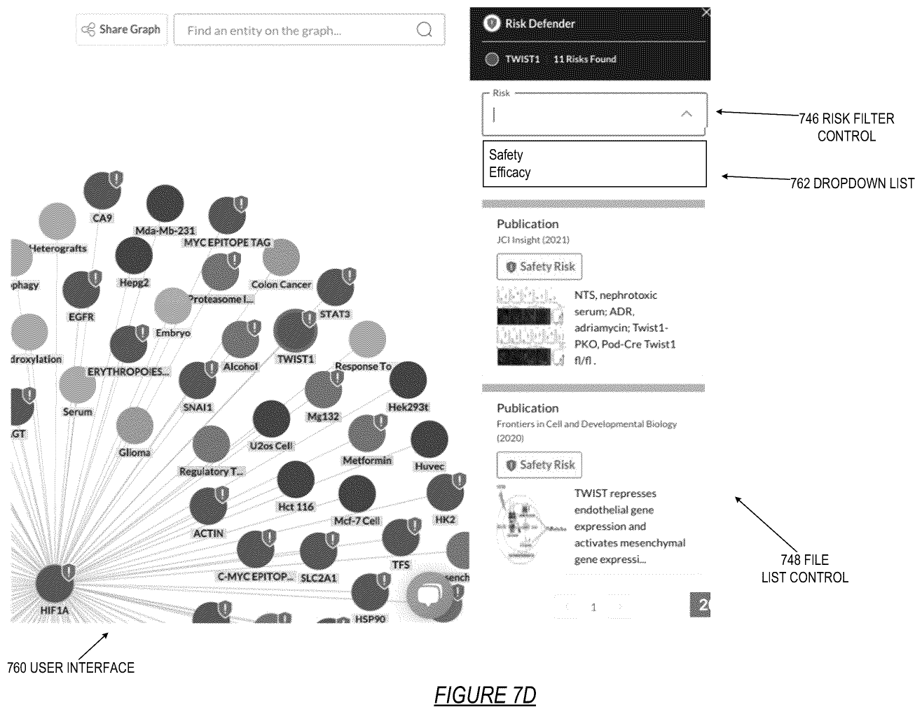

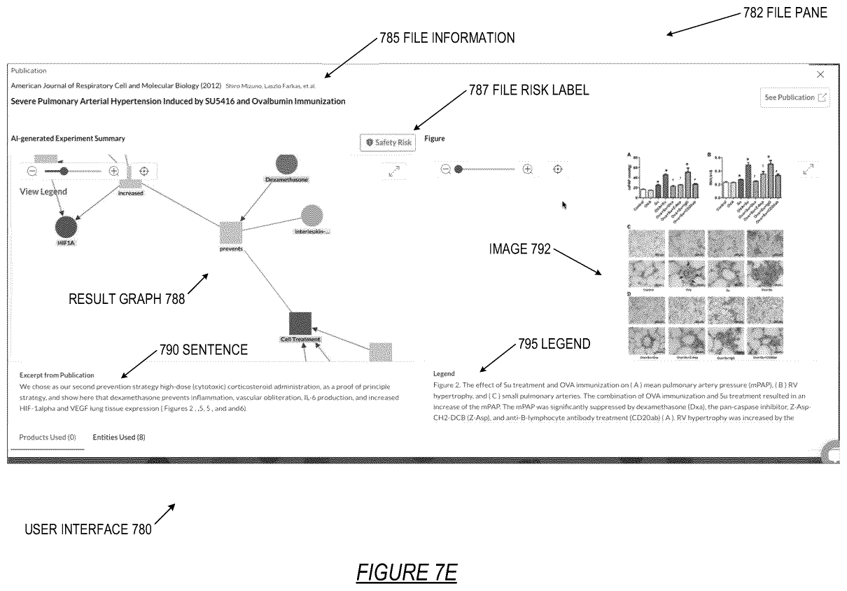

FIGS. 7A through 7E illustrate user interfaces expose risk types of files (sources of biomedical information, i.e., biomedical sources) using nodes, evidence graphs, and windows. In one embodiment, the nodes, evidence graphs, and windows may be generated by a server and displayed on a user device.

Turning to FIG. 7A, the user interface (700) displays the evidence graph (702). The user interface (700) may be displayed on a user device (a personal computer, a smartphone, a tablet, etc.).

The evidence graph (702) is generated from processing result graphs that were generated from processing files (biomedical sources). The evidence graph (702) includes the central node (704) and multiple surrounding nodes that are connected by edges. The surrounding nodes include the expanded node (706) and the selected node (708). The presence of an edge between two nodes indicates that there is a file from which a result graph is generated that includes the entities identified by the nodes of the edge.

The expanded node (706) is one of these surrounding nodes of the central node (704). The expanded node (706) was previously selected and expanded.

The selected node (708), which is displayed with the risk icon (709), has been selected by the user. The selected node (708) may be selected by hovering over the node for a threshold period of time, clicking on the selected node (708), tapping on the selected node (708), etc. Selection of the selected node (708) updates the user interface (700) to include the node highlight (710) and the popup window (712).

The node highlight (710) surrounds the selected node. The node highlight (710) indicates that the selected node (708) has been selected.

The selected node (708) is displayed with the risk icon (709). The risk icon (709) indicates that one of the experiments represented by the edge between the selected node (708) and the central node (704) includes a risk event.

The popup window (712) displays information corresponding to the selected node (708) and includes multiple user interface elements that expose additional functionality. The information displayed in the popup window (712) includes the node description (713). The user interface elements include selection elements that, when selected, trigger the additional functionality. The selection elements include the expand button (714), the risk button (715), the view all button (716), the filter button (717), and the new search button (718).

The node description (713) displays information about the entity corresponding to selected node (708). The selected node (708) represents the entity identified as “TWIST1”. Different nodes may be represented with different colors in the evidence graph (702) to indicate the type of entity to which a node corresponds. For example, the selected node (708) may be displayed with a blue color that indicates that the selected nodes of 100 represents an entity that is a gene that codes a protein. The node description (713) also includes text that identifies the existence of multiple aliases for the entity. In one embodiment, hovering over the text describing the aliases may display a window that displays the names of the aliases.

The expand button (714) is a button in the popup window (712). The expand button (714) will expand the evidence graph to include additional nodes representing entities for which a result graph includes entities represented by both the selected node and the additional node but may not include the entity represented by the central node (704). The expanded node (706) shows an example of how an expansion may be displayed with the evidence graph (702).

The risk button (715) is a button on the popup window (712). The presence of the risk button (715) in the popup window (712) indicates that at least one of the experiments and files corresponding to the edge between the selected node (708) and the central node (704) includes a risk event (e.g., a safety risk, an efficacy risk, etc.). The risk button (715) is context sensitive and includes an enumeration of the number of experiments for which a risk event was detected. For example, the risk button (715) indicates that “11” risks were found in the experiments and files of the edge between the selected node (708) and the central node (704). Selection of the risk button (715) may transition the user interface (700) to the user interface (740) of FIG. 7C.

The view all button (716) is a button on the popup window (712). Selection of the view all button (716) may transition the user interface (700) to the user interface (720) of FIG. 7B.

The filter button (717) is a button on the popup window (712). Selection of the filter button (717) filters the evidence graph (702) to remove some of the surrounding nodes. The surrounding nodes removed are surrounding nodes in which the edges (i.e., result graphs, experiments, and files) connecting the central node (704) to the surrounding nodes do not include the entity represented by the selected node (708) (e.g., “TWIST1”).

The new search button (718) is a button on the popup window (712). Selection of the new search button (718) generates a new evidence graph that has the entity of the selected node (708) as the central node of the new evidence graph.

Turning to FIG. 7B, the user interface (720) is updated to display the edge window (722). The user interface (720) may be displayed on a user device (a personal computer, a smartphone, a tablet, etc.).

The edge window (722) is displayed after selection of the view all button (716) of FIG. 7A. The edge window (722) includes the edge description (724), the risk filter control (726), the export control (727), and the file list control (728).

The edge description (724) describes the edge between the central node (704) and the selected node (708). The central node (704) corresponds to the entity identified as “HIF1A”, and the selected node (708) corresponds to the entity identified as “TWIST1”. The edge description (724) indicates that there are “102 Experiments” and indicates that there are 102 result graphs generated from files that include the entities identified as “HIF1A” and “TWIST1”.

The risk filter control (726) is a dropdown box of the edge window (722). Interaction with the risk filter control (726) allows for a selection of the risk types to be used to filter the files shown in the file list control (728). As shown, the risk filter control (726) has not been used to filter the files of the file list control (728) so that the file list control (728) may display files that do not include risk events.

The export control (727) is a button of the edge window (722). Interaction with the export control (727) exports the files identified for the edge of the edge window (722). In response to interaction with the export control (727), descriptions of files that may be viewed in the file list control (728) may be exported to a structured file. For example, the structured file may be a common separated value (CSV) file that includes a table with rows for each file and columns for data from the files. The columns may include the title of the publication for the file, the title of the article, the date of publication, etc.

The file list control (728) is a control of the edge window (722). The file list control (728) displays the files that correspond to the edge between the central node (704) and the selected node (708). The files are displayed with file tiles, which include the file tile (729).

The file tile (729) is a tile of the file list control (728). The file tile (729) displays information about a file from which a result graph was generated that includes the entities identified by the central node (704) and the selected node (708). The file tile (729) includes the name of the publication for the file, an image from the file, and a summary that corresponds to the file. The image is from a figure of the file that corresponds to the result graph generated for the file. The summary is text that may have been extracted from the file or generated from the result graph generated for the file.

Turning to FIG. 7C, the user interface (740) is updated to display the risk window (742) in response to selection of the risk button (715) (of FIG. 7A). The user interface (740) may be displayed on a user device (a personal computer, a smartphone, a tablet, etc.).

The risk window (742) is displayed after selection of the risk button (715) of FIG. 7A. The risk window (742) includes the node risk description (744), the risk filter control (746), and the file list control (748).

The node risk description (744) displays information about the risk events related to the entity (“TWIST1”) of the selected node (708). The node risk description (744) indicates that 11 risks have been found with the entity “TWIST1” for the evidence graph (702).

The risk filter control (746) is a dropdown box of the risk window (742). Interaction with the risk filter control (746) allows for a selection of the risk types to be used to filter the files shown in the file list control (748).

The file list control (748) is a control of the risk window (742). The file list control (748) displays the files that correspond to the selected node (708). The files are displayed with file tiles, which include the file tile (750).

The file tile (750) is a tile of the file list control (748). The file tile (750) displays information about a file from which a result graph was generated that includes the entities identified by the central node (704) and the selected node (708). The file tile (750) includes the summary risk label (752). The file tile (750) also includes the name of the publication of the file, and image from the file, and a summary that corresponds to the file. The image is from a figure of the file that corresponds to the result graph generated for the file. The summary is text that may have been extracted from the file or generated from the result graph generated for the file.

The summary risk label (752) is an element within the file tile (750). The presence of the summary risk label (752) indicates that a risk event was detected for the experiment described in the image of the file tile (750).

Turning to FIG. 7D, the user interface (760) is updated to display the dropdown list (762) in response to selection of the risk filter control (746) (of FIG. 7C). The user interface (760) may be displayed on a user device (a personal computer, a smartphone, a tablet, etc.).

The dropdown list (762) is displayed after a user selects the risk filter control (746), e.g., by clicking on the risk filter control (746). The dropdown list (762) displays two types of risks “Safety” and “Efficacy” for which the files in the file list control (748) may the filtered.

Turning to FIG. 7E, the user interface (780) is updated. The user interface (780) is updated to display the file pane (782).

The file pane (782) is displayed in response to selection of the file tile (750) (of FIG. 7C). The file pane (782) displays the file information (785), the file risk label (787), the result graph (788), the sentence (790), the image (792), and the legend (795).

The file information (785) describes the file from which the result graph (788) is generated. The file information (785) includes the name of the publication of the file, the date of the publication, and the title of the article from the publication.

The file risk label (787) displayed on the file pain (782). The presence of the file risk level (787) indicates that a risk event was identified from either the result graph (788) or the underlying file. The file risk label (787) identifies the type of risk as a “Safety risk”.

The result graph (788) is displayed in the file pane (782). The result graph (788) is generated from the sentence (790).

The image (792) is displayed in the file pane (782). The image (792) corresponds to the sentence (790) and is referenced in the text of the sentence (790). The legend (795) is the text that describes the image (792).

Embodiments of the invention may be implemented on a computing system. Any combination of a mobile, a desktop, a server, a router, a switch, an embedded device, or other types of hardware may be used. For example, as shown in FIG. 8A, the computing system (800) may include one or more computer processor(s) (802), non-persistent storage (804) (e.g., volatile memory, such as a random access memory (RAM), cache memory), persistent storage (806) (e.g., a hard disk, an optical drive such as a compact disk (CD) drive or a digital versatile disk (DVD) drive, a flash memory, etc.), a communication interface (812) (e.g., Bluetooth interface, infrared interface, network interface, optical interface, etc.), and numerous other elements and functionalities.

The computer processor(s) (802) may be an integrated circuit for processing instructions. For example, the computer processor(s) (802) may be one or more cores or micro-cores of a processor. The computing system (800) may also include one or more input device(s) (810), such as a touchscreen, a keyboard, a mouse, a microphone, a touchpad, an electronic pen, or any other type of input device.

The communication interface (812) may include an integrated circuit for connecting the computing system (800) to a network (not shown) (e.g., a local area network (LAN), a wide area network (WAN) such as the Internet, a mobile network, or any other type of network) and/or to another device, such as another computing device.

Further, the computing system (800) may include one or more output device(s) (808), such as a screen (e.g., a liquid crystal display (LCD), a plasma display, a touchscreen, a cathode ray tube (CRT) monitor, a projector, or other display device), a printer, an external storage, or any other output device. One or more of the output device(s) (808) may be the same or different from the input device(s) (810). The input and output device(s) (810 and (808)) may be locally or remotely connected to the computer processor(s) (802), non-persistent storage (804), and persistent storage (806). Many different types of computing systems exist, and the aforementioned input and output device(s) (810 and (808)) may take other forms.

Software instructions in the form of computer readable program code to perform embodiments of the invention may be stored, in whole or in part, temporarily or permanently, on a non-transitory computer readable medium such as a CD, a DVD, a storage device, a diskette, a tape, flash memory, physical memory, or any other computer readable storage medium. Specifically, the software instructions may correspond to computer readable program code that, when executed by a processor(s), is configured to perform one or more embodiments of the invention.

The computing system (800) in FIG. 8A may be connected to or be a part of a network. For example, as shown in FIG. 8B, the network (820) may include multiple nodes (e.g., node X (822), node Y (824)). Each node may correspond to a computing system, such as the computing system (800) shown in FIG. 8A, or a group of nodes combined may correspond to the computing system (800) shown in FIG. 8A. By way of an example, embodiments of the invention may be implemented on a node of a distributed system that is connected to other nodes. By way of another example, embodiments of the invention may be implemented on a distributed computing system having multiple nodes, where each portion of the invention may be located on a different node within the distributed computing system. Further, one or more elements of the aforementioned computing system (800) may be located at a remote location and connected to the other elements over a network.

Although not shown in FIG. 8B, the node may correspond to a blade in a server chassis that is connected to other nodes via a backplane. By way of another example, the node may correspond to a server in a data center. By way of another example, the node may correspond to a computer processor or micro-core of a computer processor with shared memory and/or resources.

The nodes (e.g., node X (822), node Y (824)) in the network (820) may be configured to provide services for a client device (826). For example, the nodes may be part of a cloud computing system. The nodes may include functionality to receive requests from the client device (826) and transmit responses to the client device (826). The client device (826) may be a computing system, such as the computing system (800) shown in FIG. 8A. Further, the client device (826) may include and/or perform all or a portion of one or more embodiments of the invention.

The computing system (800) or group of computing systems described in FIGS. 8A and 8B may include functionality to perform a variety of operations disclosed herein. For example, the computing system(s) may perform communication between processes on the same or different system. A variety of mechanisms, employing some form of active or passive communication, may facilitate the exchange of data between processes on the same device. Examples representative of these inter-process communications include, but are not limited to, the implementation of a file, a signal, a socket, a message queue, a pipeline, a semaphore, shared memory, message passing, and a memory-mapped file. Further details pertaining to a couple of these non-limiting examples are provided below.

Based on the client-server networking model, sockets may serve as interfaces or communication channel end-points enabling bidirectional data transfer between processes on the same device. Foremost, following the client-server networking model, a server process (e.g., a process that provides data) may create a first socket object. Next, the server process binds the first socket object, thereby associating the first socket object with a unique name and/or address. After creating and binding the first socket object, the server process then waits and listens for incoming connection requests from one or more client processes (e.g., processes that seek data). At this point, when a client process wishes to obtain data from a server process, the client process starts by creating a second socket object. The client process then proceeds to generate a connection request that includes at least the second socket object and the unique name and/or address associated with the first socket object. The client process then transmits the connection request to the server process. Depending on availability, the server process may accept the connection request, establishing a communication channel with the client process, or the server process, busy in handling other operations, may queue the connection request in a buffer until server process is ready. An established connection informs the client process that communications may commence. In response, the client process may generate a data request specifying the data that the client process wishes to obtain. The data request is subsequently transmitted to the server process. Upon receiving the data request, the server process analyzes the request and gathers the requested data. Finally, the server process then generates a reply including at least the requested data and transmits the reply to the client process. The data may be transferred, more commonly, as datagrams or a stream of characters (e.g., bytes).

Shared memory refers to the allocation of virtual memory space in order to substantiate a mechanism for which data may be communicated and/or accessed by multiple processes. In implementing shared memory, an initializing process first creates a shareable segment in persistent or non-persistent storage. Post creation, the initializing process then mounts the shareable segment, subsequently mapping the shareable segment into the address space associated with the initializing process. Following the mounting, the initializing process proceeds to identify and grant access permission to one or more authorized processes that may also write and read data to and from the shareable segment. Changes made to the data in the shareable segment by one process may immediately affect other processes, which are also linked to the shareable segment. Further, when one of the authorized processes accesses the shareable segment, the shareable segment maps to the address space of that authorized process. Often, only one authorized process may mount the shareable segment, other than the initializing process, at any given time.

Other techniques may be used to share data, such as the various data sharing techniques described in the present application, between processes without departing from the scope of the invention. The processes may be part of the same or different application and may execute on the same or different computing system.

Rather than or in addition to sharing data between processes, the computing system performing one or more embodiments of the invention may include functionality to receive data from a user. For example, in one or more embodiments, a user may submit data via a graphical user interface (GUI) on the user device. Data may be submitted via the graphical user interface by a user selecting one or more graphical user interface widgets or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

By way of another example, a request to obtain data regarding the particular item may be sent to a server operatively connected to the user device through a network. For example, the user may select a uniform resource locator (URL) link within a web client of the user device, thereby initiating a Hypertext Transfer Protocol (HTTP) or other protocol request being sent to the network host associated with the URL. In response to the request, the server may extract the data regarding the particular selected item and send the data to the device that initiated the request. Once the user device has received the data regarding the particular item, the contents of the received data regarding the particular item may be displayed on the user device in response to the user's selection. Further to the above example, the data received from the server after selecting the URL link may provide a web page in Hyper Text Markup Language (HTML) that may be rendered by the web client and displayed on the user device.

Once data is obtained, such as by using techniques described above or from storage, the computing system, in performing one or more embodiments of the invention, may extract one or more data items from the obtained data. For example, the extraction may be performed as follows by the computing system (800) in FIG. 8A. First, the organizing pattern (e.g., grammar, schema, layout) of the data is determined, which may be based on one or more of the following: position (e.g., bit or column position, Nth token in a data stream, etc.), attribute (where the attribute is associated with one or more values), or a hierarchical/tree structure (consisting of layers of nodes at different levels of detail-such as in nested packet headers or nested document sections). Then, the raw, unprocessed stream of data symbols is parsed, in the context of the organizing pattern, into a stream (or layered structure) of tokens (where each token may have an associated token “type”).

Next, extraction criteria are used to extract one or more data items from the token stream or structure, where the extraction criteria are processed according to the organizing pattern to extract one or more tokens (or nodes from a layered structure). For position-based data, the token(s) at the position(s) identified by the extraction criteria are extracted. For attribute/value-based data, the token(s) and/or node(s) associated with the attribute(s) satisfying the extraction criteria are extracted. For hierarchical/layered data, the token(s) associated with the node(s) matching the extraction criteria are extracted. The extraction criteria may be as simple as an identifier string or may be a query presented to a structured data repository (where the data repository may be organized according to a database schema or data format, such as XML).

The extracted data may be used for further processing by the computing system. For example, the computing system (800) of FIG. 8A, while performing one or more embodiments of the invention, may perform data comparison. Data comparison may be used to compare two or more data values (e.g., A, B). For example, one or more embodiments may determine whether A>B, A=B, A !=B, A<B, etc. The comparison may be performed by submitting A, B, and an opcode specifying an operation related to the comparison into an arithmetic logic unit (ALU) (i.e., circuitry that performs arithmetic and/or bitwise logical operations on the two data values). The ALU outputs the numerical result of the operation and/or one or more status flags related to the numerical result. For example, the status flags may indicate whether the numerical result is a positive number, a negative number, zero, etc. By selecting the proper opcode and then reading the numerical results and/or status flags, the comparison may be executed. For example, in order to determine if A>B, B may be subtracted from A (i.e., A−B), and the status flags may be read to determine if the result is positive (i.e., if A>B, then A−B>0). In one or more embodiments, B may be considered a threshold, and A is deemed to satisfy the threshold if A=B or if A>B, as determined using the ALU. In one or more embodiments of the invention, A and B may be vectors, and comparing A with B requires comparing the first element of vector A with the first element of vector B, the second element of vector A with the second element of vector B, etc. In one or more embodiments, if A and B are strings, the binary values of the strings may be compared.

The computing system (800) in FIG. 8A may implement and/or be connected to a data repository. For example, one type of data repository is a database. A database is a collection of information configured for ease of data retrieval, modification, re-organization, and deletion. A Database Management System (DBMS) is a software application that provides an interface for users to define, create, query, update, or administer databases.

The user, or software application, may submit a statement or query into the DBMS. Then the DBMS interprets the statement. The statement may be a select statement to request information, update statement, create statement, delete statement, etc. Moreover, the statement may include parameters that specify data, or data container (database, table, record, column, view, etc.), identifier(s), conditions (comparison operators), functions (e.g., join, full join, count, average, etc.), sort (e.g., ascending, descending), or others. The DBMS may execute the statement. For example, the DBMS may access a memory buffer, a reference or index a file for read, write, deletion, or any combination thereof, for responding to the statement. The DBMS may load the data from persistent or non-persistent storage and perform computations to respond to the query. The DBMS may return the result(s) to the user or software application.

The computing system (800) of FIG. 8A may include functionality to present raw and/or processed data, such as results of comparisons and other processing. For example, presenting data may be accomplished through various presenting methods. Specifically, data may be presented through a user interface provided by a computing device. The user interface may include a GUI that displays information on a display device, such as a computer monitor or a touchscreen on a handheld computer device. The GUI may include various GUI widgets that organize what data is shown as well as how data is presented to a user. Furthermore, the GUI may present data directly to the user, e.g., data presented as actual data values through text, or rendered by the computing device into a visual representation of the data, such as through visualizing a data model.

For example, a GUI may first obtain a notification from a software application requesting that a particular data object be presented within the GUI. Next, the GUI may determine a data object type associated with the particular data object, e.g., by obtaining data from a data attribute within the data object that identifies the data object type. Then, the GUI may determine any rules designated for displaying that data object type, e.g., rules specified by a software framework for a data object class or according to any local parameters defined by the GUI for presenting that data object type. Finally, the GUI may obtain data values from the particular data object and render a visual representation of the data values within a display device according to the designated rules for that data object type.

Data may also be presented through various audio methods. In particular, data may be rendered into an audio format and presented as sound through one or more speakers operably connected to a computing device.

Data may also be presented to a user through haptic methods. For example, haptic methods may include vibrations or other physical signals generated by the computing system. For example, data may be presented to a user using a vibration generated by a handheld computer device with a predefined duration and intensity of the vibration to communicate the data.

The above description of functions presents only a few examples of functions performed by the computing system (800) of FIG. 8A and the nodes (e.g., node X (822), node Y (824)) and/or client device (826) in FIG. 8B. Other functions may be performed using one or more embodiments of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A method comprising:
processing a plurality of result graphs to generate an evidence graph, in response to a query;
presenting the evidence graph with a node corresponding to a result graph of the plurality of result graphs, wherein the result graph corresponds to a file; and
presenting the node with a risk icon indicating a subset of files is identified with one or more risk types,
wherein the subset of files is a subset of a plurality of files corresponding to the plurality of result graphs,
wherein the subset of files comprises the file, and
wherein the file includes a word corresponding to the node.

2. The method of claim 1, further comprising:
receiving the file;
processing the file to generate the result graph, of the plurality of result graphs,
wherein result graph corresponds to a figure from the file, and
wherein the plurality of result graphs corresponds to the plurality of files; and
processing one or more of the result graph and the file to identify the one or more risk types of the file.

3. The method of claim 1, further comprising:
receiving the query from a user device.

4. The method of claim 1, further comprising:
receiving a selection of the node; and
processing the selection of the node to present a popup window in response to selecting the node.

5. The method of claim 1, further comprising:
presenting a popup window with a selection element indicating that the subset of files, corresponding to the node, comprises one or more risk events of the one or more risk types; and
presenting the selection element with an enumeration of the subset of files.

6. The method of claim 1, further comprising:
receiving a selection of a selection element;
processing the selection of the selection element to present a risk window;
presenting the risk window with a filter control, an export control, and a file list control;
presenting the file list control with one or more file summary tiles comprising a file summary tile;
presenting the file summary tile with an image from a figure from the file, a summary sentence generated from the file, and a summary risk label identifying a risk event of the file;
receiving a selection of the file summary tile;
processing the selection of the file summary tile to present a file pane; and
presenting the file pane with the result graph, the image, a legend sentence of the figure of the image, and a file risk label.

7. The method of claim 1, further comprising:
presenting a risk window with a filter control and a file list control; and
in response to interaction with the filter control, updating the file list control using one or more selections of the one or more risk types.

8. The method of claim 1, further comprising:
presenting a risk window with an export control and a file list control; and
in response to interaction with the export control, exporting descriptions of files listed in the file list control to a structured file.

9. A method comprising:
receiving a selection of a node of an evidence graph generated from a plurality of result graphs;
processing the selection of the node to present a popup window in response to the selection of the node;
presenting the popup window with a selection element indicating presence of one or more risk types in a subset of files corresponding to the node,
wherein the subset of files is a subset of a plurality of files corresponding to the plurality of result graphs; and
presenting the selection element with an enumeration of the subset of files.

10. The method of claim 9, further comprising:
receiving the file;
processing the file to generate the result graph, of the plurality of result graphs,
wherein result graph corresponds to a figure from the file; and
processing one or more of the result graph and the file to identify the one or more risk types of the file.

11. The method of claim 9, further comprising:
receiving a query.
processing a plurality of result graphs to generate an evidence graph, in response to the query;
presenting the evidence graph with a node corresponding to a result graph of the plurality of result graphs, wherein the result graph corresponds to a file; and
presenting the node with a risk icon indicating a subset of files is identified with one or more risk types,
wherein the subset of files is a subset of the plurality of files,
wherein the subset of files comprises the file, and
wherein the file includes a word corresponding to the node.

12. The method of claim 9, further comprising:
receiving a selection of the selection element;
processing the selection of the selection element to present a risk window;
presenting the risk window with a filter control, an export control, and a file list control;
presenting the file list control with one or more file summary tiles comprising a file summary tile;
presenting the file summary tile with an image from a figure from the file, a summary sentence generated from the file, and a summary risk label identifying a risk event of the file;
receiving a selection of the file summary tile;
processing the selection of the file summary tile to present a file pane; and
presenting the file pane with the result graph, the image, a legend sentence of the figure of the image, and a file risk label.

13. The method of claim 9, further comprising:
presenting a risk window with a filter control and a file list control; and
in response to interaction with the filter control, updating the file list control using one or more selections of the one or more risk types.

14. The method of claim 9, further comprising:
presenting a risk window with an export control and a file list control; and
in response to interaction with the export control, exporting descriptions of files listed in the file list control to a structured file.

15. A system comprising:
an evidence graph controller configured to generate an evidence graph;
an interface controller configured to present a risk window; and
an application executing on one or more servers and configured for:
receiving a selection of a selection element;
processing the selection of the selection element to present the risk window adjacent to the evidence graph using the interface controller;
presenting the risk window with a filter control, an export control, and a file list control;
presenting the file list control with one or more file summary tiles comprising a file summary tile,
wherein the file summary tile corresponds to a file of a plurality of files corresponding to a plurality of result graphs used to generate the evidence graph; and
presenting the file summary tile with an image from a figure from the file, a summary sentence generated from the file, and a summary risk label identifying a risk event of the file.

16. The system of claim 15, wherein the application is further configured for:
receiving the file;
processing the file to generate the result graph, of the plurality of result graphs, wherein result graph corresponds to the figure from the file;
processing one or more of the result graph and the file to identify the one or more risk types of the file;
receiving a selection of the file summary tile;
processing the selection of the file summary tile to present a file pane; and
presenting the file pane with the result graph, the image, a legend sentence of the figure of the image, and a file risk label.

17. The system of claim 15, wherein the application is further configured for:
receiving a query;
processing a plurality of result graphs to generate an evidence graph, in response to the query;
presenting the evidence graph with a node corresponding to a result graph of the plurality of result graphs, wherein the result graph corresponds to a file; and
presenting the node with a risk icon indicating a subset of files is identified with one or more risk types,
wherein the subset of files is a subset of a plurality of files,
wherein the subset of files comprises the file, and
wherein the file includes a word corresponding to the node.

18. The system of claim 15, wherein the application is further configured for:
receiving a selection of a node; and
processing the selection of the node to present a popup window in response to selecting the node.

19. The system of claim 15, wherein the application is further configured for:
presenting a popup window with a selection element indicating that a subset of files, corresponding to a node, comprises one or more risk events of the one or more risk types, wherein the subset of files is a subset of the plurality of files; and
presenting the selection element with an enumeration of the subset of files.

20. The system of claim 15, wherein the application is further configured for:
in response to interaction with the filter control, updating the file list control using one or more selections of the one or more risk types; and
in response to interaction with the export control, exporting descriptions of files listed in the file list control to a structured file.
